(19) United States
(12) Patent Application Publication
(10) Pub. No.: US 2008/0167584 A1
(43) Pub. Date: Jul. 10, 2008

(54) TRANSDERMAL ULTRASONIC ANTIMICROBIAL TREATMENT AND METHOD

(76) Inventor: Martin L. Lenhardt, Hayes, VA (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(21) Appl. No.: 11/969,469

(22) Filed: Jan. 4, 2008

Related U.S. Application Data
(60) Provisional application No. 60/878,365, filed on Jan. 4, 2007.

(51) Int. Cl. A61N 7/00

(52) U.S. Cl. 601/2

(57) ABSTRACT

An ultrasonic transducer pair, preferentially coupled in a phase-shifted manner, is used to provide antimicrobial treatment of an infection without heating or cavitation.

### CROSS-REFERENCE TO RELATED APPLICATIONS

This application claims priority to U.S. provisional No. 60/878,365 entitled Transdermal Ultrasonic Animicrobial Treatment And Method filed Jan. 4, 2007. Its contents are hereby incorporated in their entirety by reference thereto.

### BACKGROUND OF THE INVENTION—FIELD OF INVENTION

The present invention relates to a device for treatment of bacterial infections and a method of using said device for treatment of bacterial infections.

### BACKGROUND OF THE INVENTION

With the advent of modern-day “super-bugs”, there is a present need for a device and method of treatment that can effectively eliminate or hinder the growth of antibiotic resistant bacterial infections. Staphylococcus Epidermidis (Staph) is primary a hospital-acquired infection, with mortality rate of 10-37%. It can result in increased hospital stays of 7-19 days and costs an estimated $6,000 per case. With antibiotic resistance rising, a new treatment modality would be very desirable. Presently Staph can only be treated with vancomycin and there is no vaccine. Over 75% of infections are associated with implanted biomaterial or intravascular catheters. Bacteria colonize the skin and mucosal surfaces and spread to blood in breaks in the mucosal membranes and skin. Treatment with ultrasound is particularly effective because rapid organism adaptation is not possible since the action is physical on the cell membrane; thus resistant forms to pharmaceutical treatment retain susceptibility. Current treatments utilizing ultrasound rely on cavitation and/or heating.

### SUMMARY OF THE INVENTION

The present invention is, in one or more embodiments, a device for treatment of bacterial and other infections comprising at least one ultrasonic transducer pair, in which a first transducer of a transducer pair is adapted to produce an ultrasonic signal that is phase-shifted relative to a second ultrasonic signal from a second transducer of said transducer pair, and said first transducer and said second transducer are independently amplified and orientated with respect to each other to provide a convergence of emitted ultrasonic signals from the transducer pair onto an infection site. The device may be phase-shifted from said second ultrasonic signal such that a maximum amplitude of the ultrasonic signal is matched with a minimum amplitude of the second ultrasonic signal, thereby producing a push-pull phase-matching. The present invention is also a method of treating an infection comprising the steps of radiating a first and a second ultrasonic signal onto said infection wherein said ultrasonic signals are not aligned in phase.

### DETAILED DESCRIPTION OF THE INVENTION

Low-level, low-frequency ultrasound applied directly to the body can be effectively used for the treatment of epidermal, mucosal infections and biofilms. The effect has been tested on Staphylococcus Epidermidis and antibiotic effect, not due to heating or cavitation (as determined by control testing and observation), has been observed. The levels are safe for topical treatment of skin and mucosal infections of the head, neck trunk and extremities.

The following elements are preferably used in one or more embodiments of the present invention:

An ultrasound source (with or without a pulsing mechanism), e.g., an oscillator with a range from 15 to 100 kHz; (30-50 kHz preferred);

A phase shifter to shift the phase of a second tone in reference to the first. At various phase differences, the beam is focused and more effective;

Dual channel amplifier to increase the vibration depending on the tissue mass; and

Piezoelectric transducers (preferably 2 or more to deliver the ultrasonic vibration; generally pairs that are phase-coupled are the most efficient modality. The transducers may be ceramic/aluminum bimorphs consisting of an aluminum base, cap and ring plus a ceramic disc. Other vibrators constructed of any piezoelectric type material or capable of vibrating in the ultrasonic range, can also be used. Suitable transducers are described in the provisional application filed by Martin Lenhardt entitled “Ring Transducer for Ultrasonic Hearing” filed Jan. 3, 2007 and the corresponding non-provisional by the same author entitled “Ring Transducers for Ultrasonic Hearing”, the contents of both of which are hereby incorporated by reference thereto.

The elements may interoperate as follows: A low frequency setting is selected on the source (oscillator), for example 34,000 Hz. The multiple channel amplifier then receives an input directly from the source and a second after it passes through a phase shifter. After amplification, the outputs from each channel are sent to the treatment transducer. The transducers are usually used in pairs oriented toward or parallel to each other. The phase shifter is adjusted to obtain maximal vibration at a focal depth. After treatment the amplifier is turned off and the transdermal transducers removed. Ideally, the paired transducers operate in a push-pull fashion, being 180 degrees out of sync with each other. However, because of changes in density and tissue effects, the phase may be adjusted to compensate to provide maximal effectiveness, i.e. the phasing may be other than 180 degrees.

RESULTS: Staphylococcus epidermidis population was reduced an average of 29% with application of low power ultrasound from a single transducer. The frequency range of 34-50 kHz was most effective. There is notable improvement with transdermal transducer pairs that are phase adjusted for focusing energy. The antimicrobial effect was not due to thermal or cavitation properties of ultrasound. It is hypothesized the bacterium physically resonate and the induced oscillation disrupts cell membranes as well as disrupting normal metabolic functions.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1) A device for treatment of bacterial and other infections comprising at least one ultrasonic transducer pair, in which a first transducer of a transducer pair is adapted to produce an ultrasonic signal that is phase-shifted relative to a second ultrasonic signal from a second transducer of said transducer pair, and said first transducer and said second transducer are independently amplified and orientated with respect to each other to provide a convergence of emitted ultrasonic signals from the transducer pair onto an infection site.

2) The device of claim 1 in which said ultrasonic signals are in the range of 15-100 kilohertz.

3) The device of claim 1 in which said ultrasonic signals are in the range of 35-50 kilohertz.

4) The device of claim 1 in which said ultrasonic signals are pulsed.

5) The device of claim 1 in which said ultrasonic signal is phase-shifted from said second ultrasonic signal such that a maximum amplitude of the ultrasonic signal is matched with a minimum amplitude of the second ultrasonic signal, thereby producing a push-pull phase-matching.

6) A method of treating an infection comprising the steps of radiating a first and a second ultrasonic signal onto said infection wherein said ultrasonic signals are not aligned in phase.

7) The method of claim 6 in said ultrasonic signals are in the range of 15-100 kilohertz.

8) The method of claim 6 in which said ultrasonic signals are in the range of 35-50 kilohertz.

9) The method of claim 6 in which said ultrasonic signals are pulsed.

10) The method of claim 6 in which said ultrasonic signal is phase-shifted from said second ultrasonic signal such that a maximum amplitude of the ultrasonic signal is matched with a minimum amplitude of the second ultrasonic signal, thereby producing a push-pull phase-matching.

11) The method of claim 6 in which said ultrasonic signal is phase-shifted from said second ultrasonic signal such that a maximum amplitude of the ultrasonic signal is substantially matched with a minimum amplitude of the second ultrasonic signal, thereby producing a push-pull phase-matching.
